(19) United States
(12) Patent Application Publication
(10) Pub. No.: US 2017/0049608 A1
(43) Pub. Date: Feb. 23, 2017

(54) Condom

(71) Applicant: Reckitt Benckiser (Brands) Limited, Slough (GB)

(72) Inventors: Ekkarin Chiaokun, Chachoengsao (TH); Kewalin Phaenthong, Chachoengsao (TH); Steven Foster, Hull (GB); Jordi Salvador, Granollers (ES); Jali Williams, Chachoengsao (TH); Sittichoke Yodrayub, Chachoengsao (TH)

(21) Appl. No.: 15/306,989

(22) PCT Filed: May 1, 2015
(86) PCT No.: PCT/GB2015/051285

§ 371 Date: Oct. 26, 2016

(30) Foreign Application Priority Data
May 2, 2014 (GB) 1407836.4

(51) Int. Cl. A61F 6/04; A61F 5/41

(52) U.S. Cl. CPC A61F 6/04; A61F 5/41; A61F 2005/414

(57) ABSTRACT

A condom in the form of a sheath is described which is closed at one end and open at the opposite end, the opposite end being surrounded by a bead having a minimum inner diameter of at least 32 mm and a wall thickness of greater than 2 mm. A method of manufacture is also described.

The present invention relates to a condom which is a thin sheath worn on a man's penis during sexual intercourse as a contraceptive and/or as a protection against infection.

Condoms are formed from a thin material, typically rubber latex, polyisoprene or polyurethane, and consist of a sheath which is closed at one end an open at its opposite end, the opening being surrounded by a bead. Condoms can be formed in various thicknesses ranging between 0.03-0.09 mm and are generally provided with a length of >180 mm and formed with a nominal width or diameter at a mid-point of the condom in a non-stressed condition (ie—the condom is not exposed to any stretching forces such as when in use) of between 49-58 mm. Regardless of the mid-point width condoms are generally always provided with a bead diameter of 35 mm and bead thickness of 1.6 mm.

It is well known that providing a constriction at the base of the penis helps to maintain a longer firmer erection. Rings are available for the application to the base of the penis for this purpose.

Attempts have been made to mimic this effect in the condom. For example, the Max Perform condom produced by Fair Squared has a “potency ring”. In effect, they have used the rolled bead which is a standard feature at the open end of the condom and reduced its inner diameter to 30 mm to increase the constriction on the base of the penis. Although this may have some effect improving constriction, this product is noticeably more difficult to don and remove over the head of the penis.

As similar approach is proposed in CN 101983622, this describes a bead inner diameter of 20 mm-30 mm. In addition, the thickness of the ring has been increased from a standard thickness of 1.6 mm to between 2.2 mm and 10 mm. Again, however, such a condom is difficult to don and remove.

The present invention is directed to a condom which provides enhanced constriction but which does not suffer from the above problem.

According to the present invention, there is provided a condom in the form of a sheath which is closed at one end and open at the opposite end, the opposite end being surrounded by a bead having a minimum inner diameter of at least 32 mm and a bead wall thickness of greater than 2 mm.

Thus, the approach taken by the present invention is fundamentally different from the above prior art in that the reliance is on increasing the thickness of the bead, rather than decreasing the inner diameter. As a result of this the condom remains easy to use. However, the increased thickness of the bead provides greater resistance to expansion at the base of the penis thereby still providing enhanced constriction.

The inner diameter will preferably be at least 33 mm, more preferably 34 mm. Typically the inner diameter is preferably 35 mm which is the standard inner diameter of the bead of a condom. The inner diameter of the bead of the condom is typically no greater than 40 mm, preferably less than 38 mm. Typically the inner diameter of the bead is no more than 37 mm.

The wall thickness of the bead depends to some extent on the inner diameter of the bead. A condom with a bead of smaller diameter may have a proportionally smaller bead thickness. However, the bead thickness is preferably greater than 2.5 mm and more preferably greater than 3 mm. The maximum bead thickness is largely determined by the ability of the material forming the bead to be rolled up and on the appearance of the product as formed. Again, this will depend, to some extent, on the other dimensions. It is envisaged that the bead thickness may be as great as 10 mm, but preferably less than 8 mm, such as less than 6 mm. However, the bead thickness is desirably less than 5 mm such as less than 4 mm.

Forming a condom of this type by rolling up an unrolled part of the sheath will require a far greater percentage of the unrolled sheath to be rolled up as compared to a standard condom. In a standard condom, the unrolled sheath is 240 mm long and 60 mm of this is used to form the rolled bead, leaving a condom length of 180 mm. With the present invention, preferably between 30% and 55% of the length of the unrolled sheath is rolled up to form the rolled bead. The same ratio of 30% to 55% of the length of the unrolled sheath to final finished condom would be the same irrespective of the length of the final rolled condom. Condoms may vary in length to as long as 210-215 mm, after the bead has been formed for the finished condom. This method of forming a condom is considered to be advantageous over joining a prefabricated ring to the bead of an existing unrolled condom due to convenience of manufacture, the associated cost of manufacture and the processing time, as well as avoiding any issues associated with material inter-compatibility between the prefabricated ring and the condom.

As set out above, the present invention is a combination of a minimum inner diameter and a bead thickness which are greater than those which are currently known. Preferably, the ratio of the bead thickness (T) to the minimum inner diameter (D) is 0.05-0.31, more preferably 0.06 to 0.15. It is particularly preferred that the range is 0.08-0.1.

According to a second aspect of the present invention, there is provided a condom in the form of a sheath which is closed at one end and open at the opposite end, the opposite end being surrounded by a bead, the bead being formed of between 30% and 55% of the length of the unrolled sheath which has been rolled into the bead. This preferably also has the above-mentioned dimensions.

According to a third aspect of the present invention, there is provided a method of forming a condom, the method comprising forming an unrolled sheath on a former and rolling 30% to 55% of the unrolled length of the sheath to form a rolled bead at an open end of the condom. This preferably also has the above-mentioned dimensions.

According to a fourth aspect of the invention, there is provided a condom in the form of a sheath which is closed at one end and open at the opposite end, the opposite end being surrounded by a rolled bead having a minimum inner diameter (D) and a bead thickness (T), wherein the ratio T/D is 0.05-0.1 and preferably 0.08-0.1, and wherein the minimum diameter is at least 32 mm. This preferably also has the above-mentioned dimensions.

All of the above-mentioned dimensions are for the condom in a state in which it is not subjected to any stretching forces. In other words, they are measurements for the condom as it would be in a pack, not as they would be when in use.

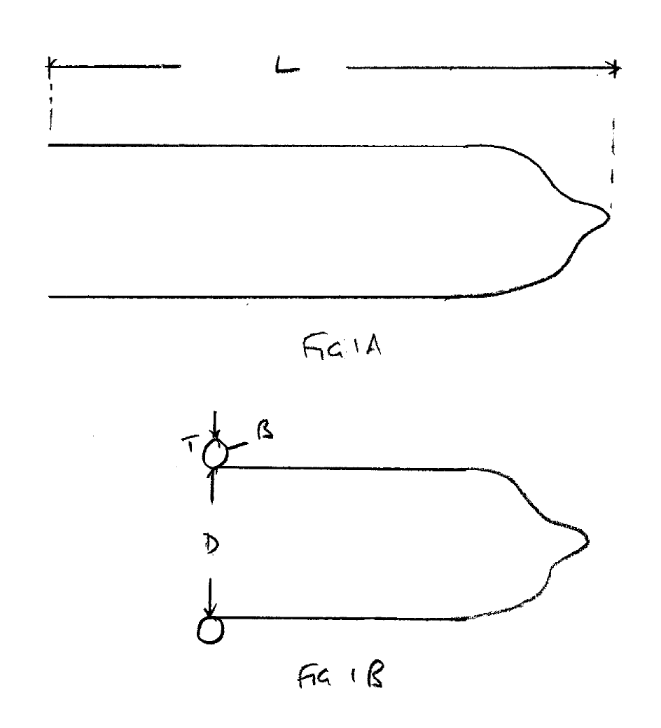

An example of a condom in accordance with the present invention will now be described with reference to FIG. 1A which is a diagrammatic cross-section of the partly formed condom and FIG. 1B which is a similar diagrammatic cross-section of a fully formed condom.

In most senses, the condom is entirely conventional. Thus it will be made of a standard condom material such as Natural Rubber Latex (NRL), Polyisoprene (PI) or Polyurethane (PU). The invention is concerned only with variations in the geometry of the bead. Thus, in all other senses, the condom may be provided with any known features of a condom including variations in the geometry of the main portion of the sheath, features provided on the wall of the sheath and known coatings.

Similarly, the condoms are generally formed by standard condom techniques. The only variation will be that a longer former is required (of 400 mm as compared to the standard of 300 mm). This former will need to be dipped deeper into the dip tank (to a depth of 350 mm as compared to the standard of 240 mm). The length of the formed sheath which then needs to be rolled to form the bead prior to vulcanisation is also increased from 170 mm as compared to the standard 60 mm.

Thus, with reference to FIG. 1A the dipped sheath has a total length L of 350 mm, 170 mm of which is rolled down on the former in a manner well known in the art to form the bead B as shown in FIG. 1B. This bead has an inner diameter D of 35 mm, which is a standard inner diameter of a condom B and a bead thickness T of 3.2 mm. This is just one example and the variations in these dimensions are set out elsewhere in the specification.

1. A condom in the form of a sheath which is closed at one end and open at the opposite end, the opposite end being surrounded by a bead haying a minimum inner diameter of at least 32 mm and a bead thickness of greater than 2 mm.

2. The condom according to claim 1, wherein the minimum inner diameter is at least 34 mm.

3. The condom according to claim 1, wherein the minimum inner diameter is at least 35 mm.

4. The condom according to claim 1, wherein the bead thickness is greater than 2.5 mm.

5. The condom according to claim 1, wherein the bead thickness is greater than 3 mm.

6. The condom according to claim 1, wherein the bead thickness is less than 5 mm.

7. The condom according to claim 1, wherein the bead thickness is less than 4 mm.

8. The condom according to claim 1 wherein bead comprises between 30% and 55% of the length of the unrolled sheath rolled up to form a rolled bead.

9. The condom according to claim 1, wherein the bead thickness is greater than the wall thickness of the rest of the condom.

10. The condom according to claim 1, wherein the bead comprise a length of the unrolled sheath rolled up to form a rolled bead, and wherein the rolled part of the bead is coated with additional material.

11. The condom according to claim 1, wherein the bead is at least partially formed as a separate component attached to a main part of the condom.

12. The condom according to claim 1, wherein the ratio of the bead thickness to the minimum inner diameter is from approximately 0.05-0.31.

13. The condom according to claim 1, wherein the ratio of the bead thickness to the minimum inner diameter is from approximately 0.06-0.15.

14. The condom according to claim 1, wherein the ratio of the bead thickness to the minimum inner diameter is from approximately 0.08-0.1

15. A condom in the form of a sheath which is closed at one end and open at the opposite end, the opposite end being surrounded by a bead, wherein the bead comprises between 30% and 55% of the length of the unrolled sheath rolled into the bead.

16. A method of forming a condom, the method comprising forming an unrolled sheath on a former and rolling 30% to 55% of the unrolled length of the sheath to form a rolled bead at an open end of the condom.

17. A condom in the form of a sheath which is closed at one end and open at the opposite end, the opposite end being surrounded by a bead having a minimum inner diameter (D) and a wall thickness (T), wherein the ratio T/D is from approximately 0.05-0.31, and wherein the minimum inner diameter (D) is at least 32 mm.

18. The condom according to claim 17, wherein the ratio T/D is from approximately 0.06-0.15.

19. The condom according to claim 17, wherein the ratio T/D is from approximately 0.08-0.1.

20. The condom according to claim 19, wherein the bead is at least partially formed as a separate component attached to a main part of the condom.
